(19) United States
(12) Patent Application Publication
(10) Pub. No.: US 2004/0242937 A1
(43) Pub. Date: Dec. 2, 2004

(54) Method for increasing the catalytic activity of multi-metal cyanide compounds

(76) Inventors: Eva Baum, Schwarzheide (DE); Georg Heinrich Grosch, Bad Durkheim (DE); Siegfried Bechtel, Lampertheim (DE); Raimund Ruppel, Dresden (DE); Kathrin Harre, Dresden (DE); Edward Bohres, Ludwigshafen (DE)

Correspondence Address: Basf Corporation, Patent Department, 1609 Biddle Avenue, Wyandotte, MI, 48192, US

(21) Appl. No.: 10/486,074

(22) PCT Filed: Aug. 10, 2002
(86) PCT No.: PCT/EP02/08988

(30) Foreign Application Priority Data
Aug. 22, 2001 (DE) 10141122.7

(51) Int. Cl. B01J 27/26; C07C 41/02

(52) U.S. Cl. 568/679; 502/175

(57) ABSTRACT

The catalytic activity of multimetal cyanide compounds used as catalysts for the addition of alkylene oxides onto H-functional starter substances is increased by subjecting the multimetal cyanide compounds to deagglomeration immediately before they are mixed with the H-functional starter substances.

The present invention relates to a method of increasing the catalytic activity of multimetal cyanide compounds which are used as catalysts in the polymerization of alkylene oxides, in particular in the preparation of polyether alcohols.

The preparation of polyether alcohols by catalytic addition of alkylene oxides onto H-functional starter substances using multimetal cyanide compounds, also known as DMC catalysts, as catalysts has been known for a long time and is described, for example, in EP-A-862,947, EP-A-892,002, EP-A-555,053 or EP-A-755,716.

The addition reaction of the alkylene oxides is generally carried out in a suspension process. For this purpose, the multimetal cyanide compounds are stirred either as powder or as a paste into the H-functional starter substances, or a catalyst suspension containing a multimetal cyanide compound is mixed with the H-functional starter substance.

Multimetal cyanide compounds are heterogeneous catalysts. This means that the available surface area and thus the number of catalytically active centers have an influence on the activity of the catalyst in the reaction.

For the industrial preparation of polyether alcohols, a very high catalytic activity of the multimetal cyanide compounds is desirable. A high catalytic activity allows a reduction in the amount of catalyst required. A high activity of the catalysts also leads to suppression of undesirable secondary reactions, for example)the formation of very high molecular weight components in the polyether alcohols, which can have an adverse effect on the properties of the desired products.

When multimetal cyanide compounds are used, agglomeration of the particles generally occurs. This agglomeration is always associated with a loss of available catalytically active surface area and thus with a reduction in the catalytic activity. This phenomenon is particularly pronounced when using multimetal cyanide compounds in the form of powder. The drying of the multimetal cyanide compounds leads to irreversible agglomeration of the particles.

If this drying step is avoided and multimetal cyanide compounds in the form of pastes or suspensions are used in place of dried powders, as described, for example, in U.S. Pat. No. 5,714,639, the agglomeration caused by drying is avoided, as shown by the reduced size of the particles. However, storage of such suspensions likewise results to an appreciable degree of undesirable agglomeration of the particles.

It is an object of the present invention to increase the catalytic activity of the multimetal cyanide compounds before use as catalysts for the addition reaction of alkylene oxides.

We have found that this object is achieved by subjecting the multimetal cyanide compounds used as catalyst for the polymerization of the alkylene oxides to deagglomeration, in particular treatment with ultrasound, and subsequently dispersing the catalyst which has been treated in this way in the H-functional starter substance for the polymerization of the alkylene oxides.

The present invention accordingly provides a method of increasing the catalytic activity of multimetal cyanide compounds for use as catalysts for the addition of alkylene oxides onto H-functional starter substances, which comprises subjecting the multimetal cyanide compounds to deagglomeration, in particular treatment with ultrasound, immediately before they are mixed with the H-functional starter substances.

The present invention further provides a process for preparing polyether alcohols by catalytic addition of alkylene oxides onto H-functional starter substances using multimetal cyanide compounds as catalysts, which comprises the steps

a) mixing at least one multimetal cyanide compound with at least one H-functional starter substance,

b) metering alkylene oxides into this mixture until the desired molecular weight of the polyether alcohol has been reached and

c) working up the polyether alcohol,

wherein the multimetal cyanide compound is subjected to deagglomeration, in particular treatment with ultrasound, immediately before it is mixed with the H-functional starter substance.

The deagglomeration of the multimetal cyanide compound is preferably carried out not more than 5 hours before the catalyst is introduced into the H-functional starter substance. Preference is given to a point in time not more than one hour before the catalyst is introduced into the H-functional starter substance. Particular preference is given to a point in time which is not more than 30 minutes before the catalyst is introduced into the H-functional starter substance. Very particular preference is given to a point in time which is not more than 5 minutes before the catalyst is introduced into the H-functional starter substance.

The point in time to be chosen for commencement of the deagglomeration is determined, inter alia, by the kinetics of the reagglomeration. If the reagglomeration kinetics are slow, as in the case of pulverulent catalysts, the treatment can be carried out some hours before preparation of the mixture of multimetal cyanide compound and H-functional starter substance. Multimetal cyanide compounds in the form of pastes or suspensions generally display rapid reagglomeration kinetics. For this reason, the deagglomeration should in this case be carried out within the last hour before preparation of the mixture of multimetal cyanide compound and H-functional starter substance.

In the case of pulverulent catalysts, deagglomeration can be carried out by milling. Here, it is advantageous to use milling apparatuses which are able to mill down to the lower micron range from 5 to 10 microns, e.g. impingement plate mills. Milling is in this case carried out on the dry multimetal cyanide compounds.

In the case of catalysts in the form of pastes and in particular in the form of suspensions, deagglomeration is carried out using dispersing systems such as bead mills, by stirring under high shear forces, e.g. wet rotor mills, and preferably by use of ultrasound. In this way, particle sizes down to 2 microns can be achieved.

Treatment with ultrasound has the advantage that deagglomeration occurs effectively and very gently. No adverse effect on the crystal structure of the multimetal cyanide compounds results.

By means of variable energy input in the treatment with ultrasound, it is possible firstly to loosen sediments. This is followed by comminution of the agglomerates, but no further than down to the primary particle size. Thus, homogenization to an ideal particle size distribution can be achieved very quickly. The desired mean particle size can be controlled by setting of the field strength, the sonication time and the mass. To achieve a very large catalytically active surface area, mean particle sizes of from 2 to 20 microns, in particular from 2 to 10 microns, are advantageous. Thus, a 400 watt ultrasound apparatus operating at 50% power can disperse 10 g of a 5% strength DMC suspension to a mean particle size of 12 microns after 3 minutes and to a mean particle size of 5 microns after 12 minutes without agglomerate residues.

After deagglomeration, the multimetal cyanide compounds are introduced into the H-functional starter substances. The multimetal cyanide compounds treated by the method of the present invention can be finely dispersed in the starter substance.

Mixing the multimetal cyanide compounds which are being treated according to the present invention into the starter substance is carried out, in particular, by use of dispersing systems such as bead mills or by vigorous stirring, if appropriate using stirrers which generate high shear forces. In the case of a suspension-like catalyst, the deagglomerated catalyst can be introduced into the H-functional starter substances by means of a reaction mixer.

The multimetal cyanide compounds are generally produced by reaction of at least one metal salt with at least one cyanometalate compound. As cyanometalate compounds, it is possible to use salts or acids. This reaction is known and described, for example, in the above-cited documents.

The multimetal cyanide compounds treated by the method of the present invention usually have the formula (I)

M¹ₐ[M²(CN)_{b}(A)_{c}]_{d}·fM¹_{g}Xₙ·h(H2O)·eL kP (I),

where

M¹ is a metal ion selected from the group consisting of Zn2+, Fe2+, Co3+, Ni2+, Mn2+, Co2+, Sn2+, Pb2+, Mo4+, Mo6+, Al3+, V4+, V5+, Sr2+, W4+, W6+, Cr2+, Cr3+, Cd2+, Hg2+, Pd2+, Pt2+, V2+, Mg2+, Ca2+, Ba2+, Cu2+,

M² is a metal ion selected from the group consisting of Fe2+, Fe3+, Co2+, Co3+, Mn2+, Mn3+, V4+, V5+, Cr2+, Cr3+, Rh3+, Ru2+, Ir3+,

and M¹ and M² are identical or different,

A is an anion selected from the group consisting of halide, hydroxide, sulfate, carbonate, cyanide, thiocyanate, isocyanate, cyanate, carboxylate, oxalate and nitrate,

X is an anion selected from the group consisting of halide, hydroxide, sulfate, carbonate, cyanide, thiocyanate, isocyanate, cyanate, carboxylate, oxalate and nitrate,

L is a water-miscible ligand selected from the group consisting of alcohols, aldehydes, ketones, ethers, polyethers, esters, polyesters, polycarbonates, ureas, amides, nitriles and sulfides,

P is an organic additive selected from the group consisting of polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkylenimines, maleic acid and maleic anhydride copolymers, hydroxyethylcellulose, polyacetates, ionic surface-active and interface-active compounds, bile acids and their salts, esters and amides, carboxylic esters of polyhydric alcohols and glycosides,

and

a, b, c, d, g and n are chosen so that the compound is electrically neutral, where c can also be 0, and

e is the number of coordination sites occupied by the ligand and is a fraction or integer greater than or equal to 0,

f is a fraction or integer greater than or equal to 0

k is a fraction or integer greater than or equal to 0

h is a fraction or integer greater than or equal to 0.

The multimetal cyanide compounds of the formula (I) can be amorphous or preferably crystalline.

The catalysts which have been activated according to the present invention can be used for preparing polyether alcohols by reacting H-functional starter substances with alkylene oxides.

In the preparation of polyether alcohols, the catalysts are used in concentrations of less than 0.1% by weight, preferably less than 500 ppm, in particular less than 250 ppm, particularly preferably less than 100 ppm, in each case based on the resulting polyether alcohol. The catalysts which have been treated according to the present invention can be used in very small amounts because of their small particle size.

H-functional starter substances employed for the preparation of polyether alcohols using the multimetal cyanide compounds which have been treated according to the present invention are, in particular, alcohols having a functionality of from 1 to 8. The functionality and the structure of the alcohols used as starters depends on the intended use of the polyether alcohols. Thus, bifunctional alcohols, in particular, are used for polyether alcohols which are to be used for producing polyurethane elastomers. For the preparation of polyether alcohols which are employed for producing flexible polyurethane foams, preference is given to using bifunctional to tetrafunctional alcohols as starter substances. To prepare polyether alcohols employed for producing rigid polyurethane foams, preference is given to using tetrafunctional to octafunctional alcohols as starter substances.

As H-functional starter substances for the preparation of polyether alcohols using the catalysts which have been treated according to the present invention, it is also possible to employ reaction products of the abovementioned alcohols with alkylene oxides, with this reaction being able to be carried out using other catalysts, in particular alkaline catalysts such as potassium hydroxide.

Examples of alcohols which can be used as H-functional starter substances for the preparation of polyether alcohols are ethylene glycol, diethylene glycol, polyethylene glycol, propylene glycol, dipropylene glycol, polypropylene glycol, glycerol, glycerol alkoxylates, trimethylolpropane, trimethylolpropane alkoxylates, pentaerythritol, glucose, sucrose.

A further class of compounds which can be prepared with the aid of the multimetal cyanide compounds which have been treated according to the present invention are addition products of alkylene oxides onto long-chain alcohols, for example fatty alcohols. Such compounds are used, for example, as surfactants.

Alkylene oxides used are usually aliphatic alkylene oxides having from 2 to 10 carbon atoms and/or styrene oxide, preferably ethylene oxide and/or propylene oxide.

The polyetherols prepared using the multimetal cyanide compounds which have been treated according to the present invention surprisingly have a reduced proportion, if any, of high molecular weight components compared to polyetherols which have been prepared using multimetal cyanide catalysts which have not been treated according to the present invention.

The reduction in the amount of high molecular weight components can very readily be seen from the viscosity of a polyether alcohol, provided that the OH number and functionality are the same as those of the polyether alcohols with which it is compared. At a low content of high molecular weight components, the viscosity of the polyether alcohols is significantly lower.

The examples below are intended to show that the treatment with ultrasound results in elimination of the DMC agglomerates, and makes the catalytically active surface more readily accessible and thus increases the catalytic activity.

The energy input also loosens the sediments and achieves dispersion down to the primary particle size. To achieve the greatest possible catalytically active surface area, preference is given to generating mean particle sizes of from 2 to 20 microns, in particular from 2 to 10 microns.

As a result of the increased catalytically active surface area, the activity of the catalyst increases and the induction time in the synthesis of the. polyether alcohols becomes shorter. The resulting polyether alcohols have lower viscosities.

### EXAMPLES

Preparation of the Double Metal Cyanide Catalyst

479.3 g of an aqueous zinc acetate solution (13.38 g of zinc acetate dihydrate and 2.2 g of Pluronic®PE 6200 (BASF AG) dissolved in 150 g of water) were heated to 50° C. While stirring with a screw stirrer, stirring energy input: 1 W/l, an aqueous hexacyanocobaltic acid solution (cobalt content: 9 g/l) and 1.5% by weight of Pluronic®PE 6200, based on the hexacyanocobaltic acid solution, were subsequently metered in over a period of 20 minutes. After all the hexacyanocobaltic acid had been introduced, the mixture was stirred for another 5 minutes at 50° C.

The temperature was subsequently reduced to 40° C. over a period of 1 hour.

The precipitated solid was separated off from the liquid by means of a pressure filter and washed with water.

The moist filter cake was subsequently dried at 50° C. in a vacuum drying oven.

The following examples were carried out using the DMC catalyst prepared in this way.

### Example 1

### Comparison

0.03 g of the DMC catalyst was added to 10 g of a polypropylene glycol having a molecular weight M_{w} of 400 g/mol, hereinafter referred to as PPG 400, and the mixture was dispersed by means of an Ultra-Turrax® T25 dispersing apparatus from IKA for 5 minutes to give a concentrate. A further 120 g of PPG400 were added and the mixture was once again homogenized for 5 minutes using the Ultra-Turrax. This PPG 400/DMC mixture was then evacuated at 3 mbar and 100° C. for 2 hours in a stirring autoclave. 70 g of propylene oxide were subsequently added at 130° C. After the increase in temperature and pressure, the maxima were determined and recorded as induction time and at the same time a measure of the activity. After the propylene oxide had reacted completely, indicated by the pressure dropping to a constant level, the contents of the autoclave were blanketed with nitrogen and the polyether alcohol was drained from the autoclave.

The results in respect of particle size, induction time and evaluation of the activity are shown in Table 1.

### Example 2

The procedure of Example 1 was repeated, but the DMC catalyst was, after the first dispersion step, deagglomerated as concentrate in PPG 400 for 3 minutes using an ultrasound generator model UP200S (200 watt) and ultrasonic probe size S14 (diameter: 14 mm) from Hilscher.

The properties of the catalyst suspension are shown in Table 1.

### Example 3

The procedure of Example 1 was repeated, but the DMC catalyst was, after the first dispersion step, deagglomerated as concentrate in PPG 400 for 1 minute in an ultrasonic bath model UTR 200 (200 watt) from Hilscher.

The induction time and analytical data for the polyether alcohol are shown in Table 1.

TABLE 1

| | | | | Evaluation |
|---|---|---|---|---|
| | Dispersing | Mean particle | Induction | of the |
| Example | apparatus | size mm | time min | activity |
| 1 | Ultra-Turrax | 18 | Did not | No reaction |
| | | | start | |
| 2 | Ultrasonic probe, | 5.6 | 3 | Very good |
| | 200 W | | | |
| 3 | Ultrasonic bath, | 6.1 | 5 | Very good |
| | 200 W | | | |

### Example 4

0.015 g of DMC catalyst was added to 10 g of PPG 400 and dispersed for 3 minutes using the ultrasound generator model UP 200S (200 watt) and ultrasonic probe S14 from Hilscher to give a concentrate. A further 120 g of PPG 400 were added and the mixture was homogenized again for 5 minutes by means of an Ultra-Turrax. The further procedure was as in Example 1.

The data and properties are shown in Table 2.

### Example 5

The procedure of Example 4 was repeated, but the DMC catalyst was deagglomerated as concentrate in PPG 400 for 6 minutes by means of the ultrasonic apparatus.

The results are shown in Table 2.

TABLE 2

| | Deagglo- | Mean | | |
|---|---|---|---|---|
| | meration | particle | Induction | Evaluation of |
| Example | time min | size mm | time min | the activity |
| 4 | 3 | 8.6 | 10 | good |
| 5 | 6 | 5.5 | 6 | very good |

### Example 6

5.0 g of DMC catalyst were added to 95 g of PPG 400 and milled in a wet rotor mill Fryma MZ 80 for 6 minutes to give a suspension. 0.4 g of this suspension were homogenized in 130 g of PPG 400 for 5 minutes by means of an Ultra-Turrax. The further procedure was as in Example 1.

The results are shown in Table 3.

### Example 7

The procedure of Example 6 was repeated, but the DMC catalyst was milled for 20 minutes.

The results are shown in Table 3.

### Example 8

The procedure of Example 7 was repeated, but the DMC catalyst was milled for 40 minutes.

The results are shown in Table 3.

TABLE 3

| | Deagglo- | Mean | | |
|---|---|---|---|---|
| | meration | particle | Induction | Evaluation of |
| Example | time min | size mm | time min | the activity |
| 6 | 6 | 8.6 | 20 | moderate |
| 7 | 20 | 6.4 | 6 | very good |
| 8 | 40 | 5.9 | 5 | very good |

### Example 9

### Polyol Synthesis using a Reduced Amount of DMC catalyst

5 g of DMC catalyst were added to 95 g of a propoxylate which was derived from glycerol and propylene oxide and had a hydroxyl number of 152 mg KOH/g and dispersed twice for 8 minutes under nitrogen using an ultrasound generator model UP1000 and ultrasonic probe S 22 from Hilscher to give a concentrate.

For the synthesis of a flexible foam polyol, 20 g of this DMC suspension were stirred into 6.3 kg of a propoxylate which was derived from glycerol and propylene oxide and had a hydroxyl number of 152 mgKOH/g under nitrogen in a 20 l stirred reactor. he contents of the reactor were treated at 110° C. under reduced pressure for 1.5 hours. 3.5 bar of nitrogen were added at 115° C., after which firstly 11.5 kg of a mixture of 9.7 kg of propylene oxide and 1.8 kg of ethylene oxide and then 2.0 kg of propylene oxide were metered in over a period of 2.5 hours. Commencement of the reaction could be observed only 10 minutes after the introduction of alkylene oxides was commenced. The reaction mixture was stirred for another 0.5 hour and then degassed at 115° C. and 8 mbar. The resulting polyether alcohol had the following properties:

Hydroxyl number: 49 mg KOH/g;

Viscosity at 25° C.: 1700 mPa s;

Zn/Co content: 13/6 ppm

### Example 10

### Comparison

5 g of DMC catalyst were added as in Example 9 to 95 g of a propoxylate which was derived from glycerol and propylene oxide and had a hydroxyl number of 152 mg KOH/g and was dispersed not by means of ultrasound but instead by means of an Ultra-Turrax model T50 from IKA under nitrogen for 10 minutes to give a concentrate. The preparation of the polyether alcohol using this suspension was carried out by a method analogous to Example 9. The induction time after commencement of the addition of alkylene oxides was 15 minutes. The resulting polyether alcohol had the following properties:

Hydroxyl number: 49 mg KOH/g;

Viscosity at 25° C.: 2200 mPa s;

Zn/Co content: 13/7 ppm

1. A method of increasing the catalytic activity of multimetal cyanide compounds for use as catalysts in paste form for the addition of alkylene oxides onto H-functional starter substances, which comprises subjecting the multimetal cyanide compounds to deagglomeration immediately before they are mixed with the H-functional starter substances.

2. A method as claimed in claim 1, wherein deagglomeration is achieved by treatment with ultrasound.

3. A method as claimed in claim 1, wherein deagglomeration is carried out not more than 30 minutes before the multimetal cyanide compounds are introduced into the H-functional starter substance.

4. A method as claimed in claim 1, wherein deagglomeration is carried out not more than 5 minutes before the multimetal cyanide compounds are introduced into the H-functional starter substance.

5. A process for preparing polyether alcohols by catalytic addition of alkylene oxides onto H-functional starter substances using multimetal cyanide compounds as catalysts, which comprises the steps of
a) mixing at least one multimetal cyanide compound with at least one H-functional starter substance,
b) metering alkylene oxides into this mixture until the desired molecular weight of the polyether alcohol has been reached and working up the polyether alcohol,
wherein the multimetal cyanide compound is subjected to deagglomeration immediately before it is mixed with the H-functional starter substance.

6. A polyether alcohol prepared in accordance with the process as claimed in claim 5.

7. A process for preparing polyether alcohols comprising the steps of
a) deagglomerating a multimetal cyanide compound;
b) mixing the multimetal cyanide compound with at least one H-functional starter substance; and
c) metering alkylene oxides into this mixture until the desired molecular weight of the polyether alcohol has been reached and working up the polyether alcohol.

8. A method as claimed in claim 7, wherein the deagglomeration step is achieved by treatment of the multimetal compound with ultrasound.

9. A method as claimed in claim 7, wherein the deagglomeration step is carried out not more than 30 minutes before the multimetal cyanide compound is introduced into the H-functional starter substance.

10. A method as claimed in claim 7, wherein the deagglomeration step is carried out not more than 5 minutes before the multimetal cyanide compounds are introduced into the H-functional starter substance.

11. A polyether alcohol prepared in accordance with the process as claimed in claim 7.

12. A polyether alcohol prepared in accordance with the process as claimed in claim 8.

13. A polyether alcohol prepared in accordance with the process as claimed in claim 9.

14. A polyether alcohol prepared in accordance with the process as claimed in claim 10.

15. A method as claimed in claim 5, wherein the deagglomeration step is achieved by treatment of the multimetal compound with ultrasound.

16. A method as claimed in claim 5, wherein the deagglomeration step is carried out not more than 30 minutes before the multimetal cyanide compound is introduced into the H-functional starter substance.

17. A method as claimed in claim 5, wherein the deagglomeration step is carried out not more than 5 minutes before the multimetal cyanide compounds are introduced into the H-functional starter substance.

18. A polyether alcohol prepared in accordance with the process as claimed in claim 15.

19. A polyether alcohol prepared in accordance with the process as claimed in claim 16.

20. A polyether alcohol prepared in accordance with the process as claimed in claim 17.
